SEQUENCE LISTING (86 sequences)

SEQ ID NO: 1 - DNA, 337 bp, Artificial Sequence
  Synthetic sequence
tcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaacaaacagacaatctggtctgtttgtattatgaattccagcacactggcggccgttactagaaataattttgtttaactttaagaaggagatataaacaaacagacaatctggtctgtttgtatta

SEQ ID NO: 2 - DNA, 160 bp, Artificial Sequence
  Synthetic sequence
gctgtaacctcgaagcgtttcacttgtaacaacgattgagaacttttgtcataaaattgaaatacttggttcgcattttcgtcatccgcggtcagccgcaattctgacgaactgcccatttagctggagaacaaacagacaatctggtctgtttgtatta

SEQ ID NO: 3 - DNA, 225 bp, Artificial Sequence
  Synthetic sequence
atggcgcgttgtagaattacgtttgtagcaatgctcagcaatctttgtcatcaaacggagacatctagtttgcatttctgtcgtgcgcggtttggtcgaaatcttgccgaaatgcccgtgtagtgagagaaaattaaagagtggagtctagcaaatacaacctttacgtgtataaattctgttgagctgcaaaacaaacagacaatctggtctgtttgtattata

SEQ ID NO: 4 - DNA, 183 bp, Agrobacterium tumefaciens
cggcacgaataacaacgtcgcgtttcatcgacgtcccatcagccgcttgtgtgttcaattcttctctgtttcacttgaaacaaattgaatagatattcccgctttcaaagccatttacaaatcctctcgtgcagcctaacgccattggcgtggaacaagcgttggcacgaggaagtaagtgcg

SEQ ID NO: 5 - DNA, 185 bp, Agrobacterium tumefaciens
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggt

SEQ ID NO: 6 - DNA, 203 bp, Agrobacterium tumefaciens
ggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaat

SEQ ID NO: 7 - DNA, 259 bp, Agrobacterium tumefaciens
ccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagct

SEQ ID NO: 8 - DNA, 305 bp, Agrobacterium tumefaciens
gttcctccctttcagctcgattgtggcatcatttattgcctgctcattgcagttgaaacgcgatatccgtttcaagacccgggtatggatggtactttggagatatgagcacttgccagacttcccggtcggagacttaaaaattccgataagaggttacggacgacggaccttataagtggatcgtctagtggtggcgccgatcaaaacagttccgcccccggctttgctcaaagtagcaaagcagctttatctcgggttgcggaggattttctagaaaaccgcaattttgcaggagagaacat

SEQ ID NO: 9 - DNA, 131 bp, Agrobacterium tumefaciens
tagctgtaacctcgaagcgtttcacttgtaacaacgattgagaacttttgtcataaaattgaaatacttggttcgcattttcgtcatccgcggtcagccgcaattctgacgaactgcccatttagctggag

SEQ ID NO: 10 - DNA, 203 bp, Agrobacterium tumefaciens
tgcacttcgcgccgcgtagcaccgacaaaggggatcggtctaattaacagataggctttaaaggatcggcttttaatgtgatttctattgaaatgcgtttaacctaggagtaaccaaaagcgccttgcaacctcacccgcactgggtaatcgtcacggataaacggcaatacgtgcctcctaaccgtgacaagagactacacc

SEQ ID NO: 11 - DNA, 126 bp, Agrobacterium tumefaciens
tcatgtatgagcaaattcagcagaaacgggacgttttgaagggcaagcgacgcctgatttcgatcctgctgcactatatgtatttcaattgcgatatcgggatcaagcagctggaggaaaacgaag

SEQ ID NO: 12 - DNA, 147 bp, Agrobacterium tumefaciens
gatcgccgtcgtactgggaatagcctgcagtatggtgcagaatataggcgcgtatactgcaaatgtaacgtaaaaatcttggtcgcaatcaaaggttctcattgcggccgtatctttcgccgatcgacttttaacgaggaagaaatc

SEQ ID NO: 13 - DNA, 124 bp, Agrobacterium tumefaciens
aaaatcactcagacagcgcggcatgacggcaaacagaaatttcagttgaaacgttattcttataaccggcgcggtcttatttgatattgtggtcggatcgcaagatttccgcggagtgagatat

SEQ ID NO: 14 - DNA, 173 bp, Agrobacterium tumefaciens
gatagctcggcaatgacgatttgattcgttcgttacagatgaaacccgattctaatgtgtaagactattttttgttgctaccgtggggcgagagcagtcgcataggctaagcctttaagtcttattgccatttacgtcgtctctcgcgtcgtctttggtgaaggtatattgca

SEQ ID NO: 15 - DNA, 152 bp, Agrobacterium tumefaciens
agtaccgcaatgccgcacagcctgcctcggcagaagcatgttacaactgaaatcaaatgcggcgaacctgaaataacccgttaactcacctcttacaagattctgcgagcgaagcgatatctgttggctcactagacttggcaggagctgtc

SEQ ID NO: 16 - DNA, 145 bp, Agrobacterium tumefaciens
agcgctcgaggtcgaaaattacaaaacatttgatagaattgcaattgtacgcagggacggtgaaacgtgttacgacaatgaacaagctctagcaaataacggagaatcgatttggggccgggagccaggcgcgttaagggcattg

SEQ ID NO: 17 - DNA, 495 bp, Agrobacterium tumefaciens
ctagcaatgcatgttggaaaaaaccgcgtttcaactgaaacactgaatttattttggcaggttccgagcagaaacaaaaagcaaggctacatacttcgccggcgttccaaacgttcgttaaggtggtcggtctcctttgttccaccgagttcgcatgaggggtcgaaagatggcgtgctcgatttgatggtcgaactgctgcaaattccgtctttggcctagcgcgccgtcacaaaggtggtgcgcgttgactcaccagatacttcttagccgtgtgattgaattcggtagcgttgagtaacgagggatgtcgctcccagcataccccggcagcaaaccgatcgcgaatgttaccgttgaaacacagtttctgtttaaccagcattcagggagtgatagaccggaagcgagatcgccgccaacaggagcaaccctagtattcctgatatatttttgatggaaatatggcgtgcatacgaaatatggaaagtatga

SEQ ID NO: 18 - DNA, 239 bp, Agrobacterium tumefaciens
cagatacttcttagccgtgtgattgaattcggtagcgttgagtaacgagggatgtcgctcccagcataccccggcagcaaaccgatcgcgaatgttaccgttgaaacacagtttctgtttaaccagcattcagggagtgatagaccggaagcgagatcgccgccaacaggagcaaccctagtattcctgatatatttttgatggaaatatggcgtgcatacgaaatatggaaagtatga

SEQ ID NO: 19 - DNA, 187 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtat

SEQ ID NO: 20 - DNA, 189 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatat

SEQ ID NO: 21 - DNA, 191 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatatat

SEQ ID NO: 22 - DNA, 193 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatatatat

SEQ ID NO: 23 - DNA, 195 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatatatatat

SEQ ID NO: 24 - DNA, 197 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatatatatatat

SEQ ID NO: 25 - DNA, 199 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatatatatatatat

SEQ ID NO: 26 - DNA, 201 bp, Artificial Sequence
  Synthetic sequence
gatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatatatatatatatat

SEQ ID NO: 27 - DNA, 185 bp, Artificial Sequence
  Synthetic sequence
gatgcgcggttctcggtccatgttgcgttccaagacgccgggcgaggtttctcactccaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 28 - DNA, 184 bp, Artificial Sequence
  Synthetic sequence
gatgcgcggttctcggtccatgttgcgttccaagacgccgggcgaggtttctcgctgcaattggaatcataaagaagcaattgaaaattttcgagtaaccgaccctccgataatcttcaacataaaacaacgcacttcttccaacgggagaggcggtgttagttgcgagctaaggagataaggt

SEQ ID NO: 29 - DNA, 185 bp, Artificial Sequence
  Synthetic sequence
gatgcgcggttctcggtccatgttgcgttccaagacgccgggcgaggtttctcgctccaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 30 - DNA, 185 bp, Artificial Sequence
  Synthetic sequence
gatgcgcggttctcggtccatgttgcgttccaagacgccgggcgaggtttctcgctgcaattggactcataaagaagcaattgaaaattttcgagtaaccgaccctcccgataatcttcaacataaaacaacgcacttcttccaacgggagaggcggtgttagttgcgagctaaggagataaggt

SEQ ID NO: 31 - DNA, 185 bp, Artificial Sequence
  Synthetic sequence
gatgcgcggttctcggtccatgttgcgttccaagacgccgggcgaggtttctcactgcaattggaaccataaagaagcaattgaaaattttcgagtaaccgaccctcccgataatcttcaacataaaacaacgcacttcttccaacgggagaggcggtgttagttgcgagctaaggagataaggt

SEQ ID NO: 32 - DNA, 185 bp, Artificial Sequence
  Synthetic sequence
gatgcgcggttctcggtccatgttgcgttccaagacgccgggcgaggtttctcgcttcaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 33 - DNA, 185 bp, Artificial Sequence
  Synthetic sequence
gatgcgcggttctcggtccatgttgcgttccaagacgccgggcgaggtttctcactgcaattgcaaccataaagaagcaattgaaaattttcgagtaaccgaccctcccgataatcttcaacataaaacaacgcacttcttccaacgggagaggcggtgttagttgcgagctaaggagataaggt

SEQ ID NO: 34 - DNA, 185 bp, Artificial Sequence
  Synthetic sequence
gatgcgcggttctcggtccatgttgcgttccaagacgccgggcgaggtttctcattccaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 35 - DNA, 185 bp, Artificial Sequence
  Synthetic sequence
gatgcgcggttctcggtccatgttgcgttccaagacgccgggcgaggtttctcattgcaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 36 - DNA, 185 bp, Artificial Sequence
  Synthetic sequence
gatgcgcggttctcggtccatgttgcgttccaagacgccgggcgaggtttctcgttgcaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 37 - DNA, 185 bp, Artificial Sequence
  Synthetic sequence
gatgcgcggttctcggtccatgttgcgttccaagacgccgggcgaggtttctcactacaattggaatcataaagaagcaattgaaaattttcgagtaaccgaccctcccgataatcttcaacataaaacaacgcacttcttccaacgggagaggcggtgttagttgcgagctaaggagataaggt

SEQ ID NO: 38 - DNA, 185 bp, Artificial Sequence; identical to SEQ ID NO: 35
  Synthetic sequence

SEQ ID NO: 39 - DNA, 185 bp, Artificial Sequence
  Synthetic sequence
gatgcgcggttctcggtccatgttgcgttccaagacgccgggcgaggtttctcattacaattggaaccataaagaagcaattgaaaattttcgagtaaccgaccctcccgataatcttcaacataaaacaacgcacttcttccaacgggagaggcggtgttagttgcgagctaaggagataaggt

SEQ ID NO: 40 - DNA, 185 bp, Artificial Sequence
  Synthetic sequence
gatgcgcggttctcggtccatgttgcgttccaagacgccgggcgaggtttctcattgcaattgtaatcataaagaagcaattgaaaattttcgagtaaccgaccctcccgataatcttcaacataaaacaacgcacttcttccaacgggagaggcggtgttagttgcgagctaaggagataaggt

SEQ ID NO: 41 - DNA, 185 bp, Artificial Sequence
  Synthetic sequence
gatgcgcggttctcggtccatgttgcgttccaagacgccgggcgaggtttctcgttacaattgaaatcataaagaagcaattgaaaattttcgagtaaccgaccctcccgataatcttcaacataaaacaacgcacttcttccaacgggagaggcggtgttagttgcgagctaaggagataaggt

SEQ ID NO: 42 - DNA, 185 bp, Artificial Sequence
  Synthetic sequence
gatgcgcggttctcggtccatgttgcgttccaagacgccgggcgaggtttctcattgcaattgtaaccataaagaagcaattgaaaattttcgagtaaccgaccctcccgataatcttcaacataaaacaacgcacttcttccaacgggagaggcggtgttagttgcgagctaaggagataaggt

SEQ ID NO: 43 - DNA, 185 bp, Artificial Sequence; identical to SEQ ID NO: 39
  Synthetic sequence

SEQ ID NO: 44 - DNA, 185 bp, Artificial Sequence
  Synthetic sequence
gatgcgcggttctcggtccatgttgcgttccaagacgccgggcgaggtttctcattacaattgtaatcataaagaagcaattgaaaattttcgagtaaccgaccctcccgataatcttcaacataaaacaacgcacttcttccaacgggagaggcggtgttagttgcgagctaaggagataaggt

SEQ ID NO: 45 - DNA, 185 bp, Artificial Sequence
  Synthetic sequence
gatgcgcggttctcggtccatgttgcgttccaagacgccgggcgaggtttctcattacaattgcaatcataaagaagcaattgaaaattttcgagtaaccgaccctcccgataatcttcaacataaaacaacgcacttcttccaacgggagaggcggtgttagttgcgagctaaggagataaggt

SEQ ID NO: 46 - DNA, 185 bp, Artificial Sequence
  Synthetic sequence
gatgcgcggttctcggtccatgttgcgttccaagacgccgggcgaggtttctcattgcaattgaaatcataaagaagcaattgaaaattttcgagtaaccgaccctcccgataatcttcaacataaaacaacgcacttcttccaacgggagaggcggtgttagttgcgagctaaggagataaggt

SEQ ID NO: 47 - DNA, 14 bp, Agrobacterium tumefaciens
rttdcawwtghaay

SEQ ID NO: 48 - DNA, 14 bp, Agrobacterium tumefaciens
gtttcacttgaaac

SEQ ID NO: 49 - DNA, 14 bp, Agrobacterium tumefaciens
gcttcaattgaaat

SEQ ID NO: 50 - DNA, 14 bp, Agrobacterium tumefaciens
gctacatttgcaat

SEQ ID NO: 51 - DNA, 14 bp, Agrobacterium tumefaciens
attgcaaatgtagc

SEQ ID NO: 52 - DNA, 14 bp, Agrobacterium tumefaciens
attgcagttgaaac

SEQ ID NO: 53 - DNA, 14 bp, Agrobacterium tumefaciens
gtttcacttgtaac

SEQ ID NO: 54 - DNA, 14 bp, Agrobacterium tumefaciens
gttacagatgaaac

SEQ ID NO: 55 - DNA, 14 bp, Agrobacterium tumefaciens
gtttcaactgaaac

SEQ ID NO: 56 - DNA, 14 bp, Agrobacterium tumefaciens
attgcaattgtacg

SEQ ID NO: 57 - DNA, 14 bp, Agrobacterium tumefaciens
gtttcaactgaaac

SEQ ID NO: 58 - DNA, 14 bp, Agrobacterium tumefaciens
gttaccgttgaaac

SEQ ID NO: 59 - DNA, 14 bp, Agrobacterium tumefaciens
atggcagttgaaat

SEQ ID NO: 60 - DNA, 14 bp, Agrobacterium tumefaciens
atttcaattgcgat

SEQ ID NO: 61 - DNA, 14 bp, Agrobacterium tumefaciens
actgcaaatgtaac

SEQ ID NO: 62 - DNA, 14 bp, Agrobacterium tumefaciens
atttcagttgaaac

SEQ ID NO: 63 - DNA, 6 bp, Agrobacterium tumefaciens
caattg

SEQ ID NO: 64 - DNA, 14 bp, Artificial Sequence
  Synthetic sequence
  misc_feature (4)..(4): n is a, c, g, or t
  misc_feature (11)..(11): n is a, c, g, or t
rytncaattgnaay

SEQ ID NO: 65 - DNA, 14 bp, Artificial Sequence
  Synthetic sequence
actccaattggaat

SEQ ID NO: 66 - DNA, 14 bp, Artificial Sequence
  Synthetic sequence
gctgcaattggaat

SEQ ID NO: 67 - DNA, 14 bp, Artificial Sequence
  Synthetic sequence
gctccaattggaat

SEQ ID NO: 68 - DNA, 14 bp, Artificial Sequence
  Synthetic sequence
gctgcaattggact

SEQ ID NO: 69 - DNA, 14 bp, Artificial Sequence
  Synthetic sequence
actgcaattggaac

SEQ ID NO: 70 - DNA, 14 bp, Artificial Sequence
  Synthetic sequence
gcttcaattggaat

SEQ ID NO: 71 - DNA, 14 bp, Artificial Sequence
  Synthetic sequence
actgcaattgcaac

SEQ ID NO: 72 - DNA, 14 bp, Artificial Sequence
  Synthetic sequence
attccaattggaat

SEQ ID NO: 73 - DNA, 14 bp, Artificial Sequence
  Synthetic sequence
attgcaattggaat

SEQ ID NO: 74 - DNA, 14 bp, Artificial Sequence
  Synthetic sequence
gttgcaattggaat

SEQ ID NO: 75 - DNA, 14 bp, Artificial Sequence
  Synthetic sequence
actacaattggaat

SEQ ID NO: 76 - DNA, 14 bp, Artificial Sequence
  Synthetic sequence
attgcaattggaat

SEQ ID NO: 77 - DNA, 14 bp, Artificial Sequence
  Synthetic sequence
attacaattggaac

SEQ ID NO: 78 - DNA, 14 bp, Artificial Sequence
  Synthetic sequence
attacaattgtaat

SEQ ID NO: 79 - DNA, 14 bp, Artificial Sequence
  Synthetic sequence
attgcaattgtaat

SEQ ID NO: 80 - DNA, 14 bp, Artificial Sequence
  Synthetic sequence
gttacaattgaaat

SEQ ID NO: 81 - DNA, 14 bp, Artificial Sequence
  Synthetic sequence
attgcaattgtaac

SEQ ID NO: 82 - DNA, 14 bp, Artificial Sequence
  Synthetic sequence
attacaattggaac

SEQ ID NO: 83 - DNA, 14 bp, Artificial Sequence
  Synthetic sequence
attacaattgtaat

SEQ ID NO: 84 - DNA, 14 bp, Artificial Sequence
  Synthetic sequence
attacaattgcaat

SEQ ID NO: 85 - DNA, 14 bp, Artificial Sequence
  Synthetic sequence
attgcaattgaaat

SEQ ID NO: 86 - DNA, 14 bp, Agrobacterium tumefaciens
gcttcaattgaaat

(19) United States
(12) Patent Application Publication
(10) Pub. No.: US 2022/0411811 A1
(43) Pub. Date: Dec. 29, 2022

(54) SYNTHETIC TOOLKIT FOR PLANT TRANSFORMATION

(71) Applicant: The Board of Trustees of the University of Illinois, Urbana, IL (US)

(72) Inventors: Ting Lu, Savoy, IL (US); Yuanchao Qian, Champaign, IL (US); Wentao Kong, Urbana, IL (US)

(21) Appl. No.: 17/849,538

(22) Filed: Jun. 24, 2022

Related U.S. Application Data
(60) Provisional application No. 63/215,249, filed on Jun. 25, 2021.

(51) Int. Cl. C12N 15/82; C12N 15/74

(52) U.S. Cl. CPC C12N 15/8238; C12N 15/743

(57) ABSTRACT

The disclosure provides a synthetic biology toolkit that enables precise and effective control of gene expression in A. tumefaciens and related Rhizobia. Inducible expression systems were constructed, characterized, and optimized to obtain an expression system regulated through amplifier introduction and promoter engineering, and cognate promoters were produced and evaluated. To establish a fine-tunability, a series of spacers and a promoter library were constructed to systematically modulate both translational and transcriptional rates. The application of the tools was demonstrated by coexpressing genes with altered expression levels using a single signal. The studies carried out provide precise expression tools, facilitating rational engineering of in A. tumefaciens and related Rhizobia bacteria for advanced plant biotechnological applications.

### REFERENCE TO RELATED APPLICATIONS

This application claims the benefit of U.S. provisional Application No. 63/215,249, filed Jun. 25, 2021, which is incorporated herein by reference in its entirety.

### INCORPORATION OF SEQUENCE LISTING

The sequence listing that is contained in the file named “AGOE003US_ST25.txt,” which is 27 KB (measured in MS-Windows) and created on Jun. 24, 2022, is filed herewith by electronic submission and incorporated herein by reference.

### FIELD OF THE INVENTION

The disclosure relates to methods, polynucleotide constructs, and systems for controlling and enhancing gene expression in Rhizobia such as Agrobacterium tumefaciens, and improving the ability of A. tumefaciens and other Rhizobia to genetically transform cells of plants and other organisms.

### BACKGROUND OF THE INVENTION

Agrobacterium tumefaciens is a soil-borne, Gram-negative bacterium that is widely studied for its ability to transfer DNA into plants. Agrobacterium-mediated transformation (AMT) is perhaps the most versatile technology for production of genetically modified plants. AMT is also used for the transformation of filamentous fungi, green algae and human cells. Agrobacterium spp., such as A. rhizogenes, and other Rhizobia, i.e. members of the Rhizobiales, such as Rhizobium spp., Mesorhizobium spp., Sinorhizobium spp., Bradyrhizobium spp. and related species and genera beyond Agrobacterium tumefaciens have also been found to be able to genetically transform plants.

In addition to its role in genetic transformation of plant cells, A. tumefaciens has been utilized in a variety of studies. For example, it has been adopted as a well-characterized model organism for the study of plant-microbe signaling (Barton, et al., Environmental Microbiology, 20:16-29, 2018; Venturi & Fuqua, Ann Rev Phytopathol, 51:17-37, 2013), bacterial cell-to-cell communication (Faure & Lang, Agrobacterium tumefaciens. Frontiers in Plant Science, 5, 14. doi:10.3389/fpls.2014.000142014), and virulence mechanisms (Jakubowski, et al, J Bacteriol, 187:3486-3495, 2005).

However, although AMT is a valuable technology for the production of genetically modified crop plants, Agrobacterium (and related Rhizobia) is not able to genetically transform certain plant (crop) species, or does so inefficiently or in a genotype-dependent manner. Improved methods of transforming plants through AMT that address these shortcomings would therefore be a significant advance in the art.

### BRIEF DESCRIPTION OF THE FIGURES

FIG. 1. shows the characterization of six inducible systems for controllable gene expression in A. tumefaciens. (A) shows gene circuit design of the six systems. (B) shows response functions of the inducible systems.

FIG. 2. shows a cumic acid-controlled, VirG_{N54D}-amplified induction system. (A) is a diagram of the controllable gene circuit. Fluorescence levels (B) and representative bright-field and green fluorescence images (C) are shown for A. tumefaciens carrying in the induction system in the absence and presence of the inducer cumic acid (Cum). (D) shows the design of four promoter variants used to drive Vir_{N54D} expression. Fluorescence levels (E) and representative bright-field and green fluorescence images (F) of cells carrying different versions of the optimal promoters are also shown.

FIG. 3. shows a library of VirG-responsive promoters. (A) provides binding site sequences of fifteen VirG controlled promoters identified in the plasmid P_{TiBO542} including VirA binding site (SEQ ID Nos:48-62); (B) shows gene expression activity of the fifteen promoters measured by the relative GFP fluorescence levels.

FIG. 4. shows fine-tuning of gene expression through spacer engineering. (A) provides spacer design including AT repeats embedded into the spacer between the ribosome binding site and the start codon the downstream gene. (B) shows fluorescence intensity as a function of the number of AT repeats for the virB promoter. (C) shows congo red images of A. tumefaciens carrying a cumic acid-inducible, pleD expression system with different spacers. NTL4 is a control, harboring no plasmid. The strains AT0-pleD, AT6-pleD and AT8-pleD harbor the plasmids P_{virB-AT0}-pled, P_{virB-AT6}-pled, and Pv_{irB-AT8}-pled respectively. (D) shows colorimetric measures of the biofilms shown in C.

FIG. 5. shows a P_{virB} promoter library with altered expression level. (A) schematic of portions of the P_{virB} promoter. (B) shows green fluorescence expression levels of the promoter variants. (C) provides partial sequences of the promoter library comprising engineered P_{virB} variant promoters.

FIG. 6. shows altered gene co-expression with a single controller. (A) shows schematic of the co-expression system used to generate varied levels of sfgfp and mKate2 expression. HH: high expression for both sfgfp and mKate2; HL: high egfp expression and low mKate2 expression; LL: low expression for both sfgfp and mKate2. (B) shows a schematic of two constructs driving differential co-expression of pled and sfgfp. (C) shows GFP and mKate fluorescence levels for the three circuit variants in A. NTL4 is a control. (D) demonstrates cellulose (congo red) and GFP fluorescence levels of strains carrying circuits in (C). NTL4 and pleD are two controls.

### BRIEF DESCRIPTION OF THE SEQUENCE LISTING

SEQ ID Nos:1-3 Artificial cumic acid-inducible promoters.

SEQ ID Nos:4-18 VirG-controlled promoters from pTiB0542.

SEQ ID Nos:19-26 Artificial VirB promoters with various AT repeats inserted in the spacer region between the promoter and the reporter gene.

SEQ ID Nos:27-46 Artificial VirB promoters with engineered VirG binding site.

SEQ ID NO:47 Consensus VirG-binding motif.

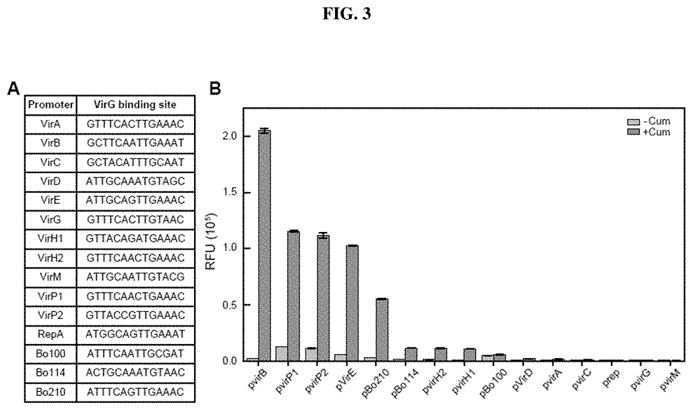

SEQ ID Nos:48-62 A. tumefaciens VirG binding sites of FIG. 3.

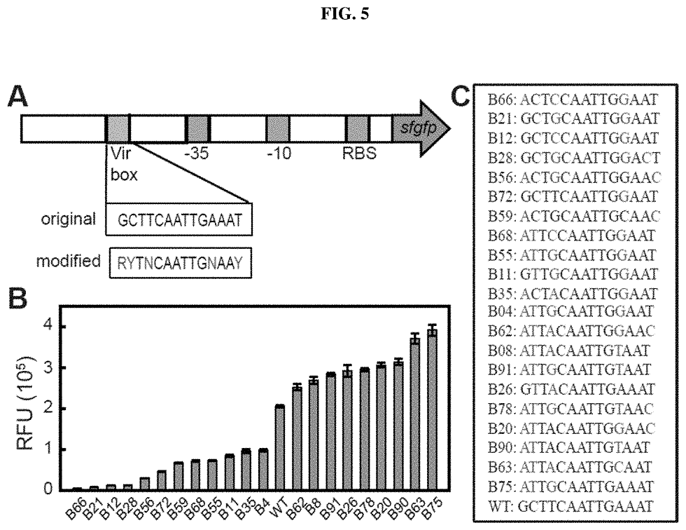

SEQ ID NO:63 P_{VirB} core region, as shown in FIG. 5.

SEQ ID NO:64 P_{VIrB} core region with consensus engineered sites.

SEQ ID Nos:65-85 Engineered P_{VirB} promoter fragments of FIG. 5C.

SEQ ID NO:86 A native WT P_{VirB} promoter fragment of FIG. 5C.

### SUMMARY OF THE INVENTION

In one aspect the invention comprises a recombinant polynucleotide construct comprising a DNA molecule encoding: (a) at least one gene of interest operably linked to a heterologous inducible promoter for expression of the gene of interest in a bacterial cell, wherein the ratio of expression of the gene of interest in the presence of an added inducer relative to expression in the absence of the added inducer is at least 100; and (b) a broad host range origin of replication functional in Enterobacteriaceae and Rhizobiaceae. In certain embodiments the inducer is cumic acid or vanillic acid. The invention also comprises embodiments wherein the origin of replication of the construct comprises an oriT functional with IncQ, IncP, IncW, or colE1. In certain embodiments the ratio of expression of the gene of interest in the presence of an added inducer relative to expression in the absence of the added inducer is at least 200, 300, 400 or 500 relative to expression in the absence of the added inducer.

In another aspect the invention comprises a transgenic bacterium comprising the recombinant polynucleotide construct comprising a DNA molecule encoding: (a) at least one gene of interest operably linked to a heterologous inducible promoter for expression of the gene of interest in a bacterial cell, wherein the ratio of expression of the gene of interest in the presence of an added inducer relative to expression in the absence of the added inducer is at least 100; and (b) a broad host range origin of replication functional in Enterobacteriaceae and Rhizobiaceae. In certain embodiments the bacterium is from a species within a genus selected from the group consisting of: Escherichia, Agrobacterium , and Rhizobium. In particular embodiments the bacterium is an Agrobacterium tumefaciens bacterium or an Agrobacterium rhizogenes bacterium. The invention may also comprise an in vitro culture of the bacterium, growing in the presence of an inducer. In some embodiments a culture of the bacterium growing in the presence both of a plant cell and of the inducer is contemplated.

In some embodiments of the invention the bacterium further comprises a VirG_{N54D} protein. In further embodiments the heterologous inducible promoter may comprise a nucleotide sequence selected from the group consisting of: SEQ ID Nos:1-3, SEQ ID Nos:19-26, and SEQ ID Nos:27-47, or the heterologous inducible promoter comprises a nucleotide sequence selected from the group consisting of: SEQ ID NO:64 and SEQ ID Nos:65-85.

In another aspect the invention provides a method for expressing a gene of interest comprising: (a) obtaining a transgenic bacterium comprising a recombinant polynucleotide construct comprising a DNA molecule encoding: (i) at least one gene of interest operably linked to a heterologous inducible promoter for expression of the gene of interest in a bacterial cell, wherein the ratio of expression of the gene of interest in the presence of an added inducer relative to expression in the absence of the added inducer is at least 100; and (ii) a broad host range origin of replication functional in Enterobacteriaceae and Rhizobiaceae wherein the heterologous inducible promoter comprises a nucleotide sequence selected from the group consisting of: SEQ ID NOs:1-3, SEQ ID Nos:19-26, SEQ ID Nos:27-47, and SEQ ID Nos:64-85; (b) growing a culture of cells of the bacterium in the presence of an inducer of the heterologous promoter; and (c) assaying the culture, or a portion or an extract thereof, for expression of the gene of interest. In such a method the culture of the bacterium may further comprise plant cells. In certain embodiments of such a method, the assaying may comprise measuring the transformation frequency (“TF”) of a plant cell by the bacterium.

The invention further provides, in another aspect, a polynucleotide construct comprising a gene of interest operably linked to a heterologous inducible promoter sequence for expression of the gene of interest in a bacterial cell, wherein the promoter sequence comprises a nucleotide sequence selected from the group consisting of: SEQ ID Nos:64-85.

A kit comprising the bacterium comprising a polynucleotide construct comprising a gene of interest operably linked to a heterologous inducible promoter sequence for expression of the gene of interest in a bacterial cell, wherein the promoter sequence comprises a nucleotide sequence selected from the group consisting of: SEQ ID Nos:1-3, 19-26, 27-46, 47, 64, and 65-85, and an inducer of the heterologous promoter is also contemplated.

### DETAILED DESCRIPTION OF THE INVENTION

Agrobacterium (and related Rhizobia) is not able to genetically transform certain plant (crop) species and certain varieties (genotypes) of other species, or does so inefficiently. Thus there is a need for enhancing the ability of A. tumefaciens and related Rhizobia to transform plant cells and other cells, especially of plant species not efficiently transformed by A. tumefaciens or other Rhizobia. The invention overcomes such limitations of the prior art by providing nucleic acid constructs, methods, and systems for enhancing and controlling gene expression and transformation by A. tumefaciens as well as other Rhizobia. In addition, the present disclosure provides methods for rational and systematic genetic engineering of bacteria to enhance plant cell transformation.

Methods and compositions for enhancement of transformation ability provided herein may include, for example, controlling and optimizing vir gene expression as well as expression of other bacteria loci, including chromosomal loci such as chv genes to achieve more efficient cell transformation including an increase in transformation frequency and an improved broader range of plant species for which efficient cell transformation is available. The approaches described herein can also allow for improved transformation efficiency of non-plant cells by Agrobacterium and other Rhizobia. The described constructs, polynucleotide sequences, and methods also provide for rationally-controlled inducible gene expression systems for expression of one or more gene(s) of interest in Rhizobia including A. tumefaciens. Expression of entire bacterial vir gene clusters and/or chromosomal chv gene clusters (or other bacterial operons) may be altered, allowing for efficient cell transformation of an expanded set of target plant (crop) or other species targeted for bacterial-mediated transformation.

Additionally, there is a need to further develop or enhance methods and polynucleotide constructs for gene expression in A. tumefactions and other Rhizobia for expression of genes of interest in a controlled manner. This may include development of effective inducible expression systems to control gene expression in Agrobacterium and other Rhizobia. As part of the described “toolkit” for gene expression described herein, the engineered inducible promoter sequences of the disclosure further allow for predictable levels of gene expression in Agrobacterium and other Rhizobia over a useful range important for fine-tuning such expression of genes (e.g. vir genes), or groups of genes such as operons, of interest. Such efficient inducible expression systems may reduce or eliminate the need for traditional phenolic inducers of Agrobacterium vir gene expression, such as acetosyringone. Reliable induction systems for gene expression may also be useful to achieve precise control of gene expression. Simple sequence repeats in the spacer region between the ribosome-binding site and the start codon (ATG) were found, for example, to effectively modulate translation in A. tumefaciens, with various lengths of AT sequence repeats ((AT)₀-(AT)₁₀) inserted in the spacer region between the promoter and the reporter gene (SEQ ID Nos:19-26) showing that altering the number of AT repeats can robustly and predictably tune gene expression levels over a 100-fold range. Since complex biosynthetic pathways often require a coordinated, fine balance of expression of individual genes in order to achieve optimal performance, the present invention allows for gene expression fine-tuning in A. tumefaciens.

The disclosure thus provides, in one embodiment, for an inducible bacterial gene expression system comprising a recombinant construct comprising a DNA molecule encoding at least one gene of interest for expression in a bacterial cell, operably linked to a heterologous inducible promoter for expression of the gene of interest in the bacterial cell, wherein the ratio of expression of the gene of interest in the presence of an added inducer relative to expression in the absence of the added inducer is at least 100×; or 500× or more relative to expression in the absence of an inducer. The efficiency of the inducible expression system may, in specific embodiments, also be measured by ascertaining the absolute level of gene expression in the presence of an inducer, relative to the expression seen in the absence of an inducer. A polynucleotide of the present invention may also comprise a broad host range origin of replication functional in both Enterobacteriaceae and Rhizobiaceae. In certain embodiments the inducer of bacterial gene expression may be cumic acid or vanillic acid. The broad host-range origin of replication may for instance comprise an oriT functional with IncQ, IncP, IncW, or colE1.

In certain embodiments, a transgenic bacterial strain comprising the recombinant construct is contemplated. The bacterial strain may be from a species of Rhizobia, or for instance from within a genus selected from the group consisting of: Escherichia, Agrobacterium, and Rhizobium. The bacterial strain may be comprised within a bacterial culture that may be growing in the presence of the inducer, or in the presence of the inducer and of a plant cell. In further embodiments the bacterial strain also comprises an “amplifier” module, such as comprising a VirG_{N54D} protein.

Also contemplated as an aspect of the invention is a polynucleotide construct comprising a gene of interest operably linked to a heterologous promoter sequence for expression of the gene of interest in a bacterial cell, wherein the promoter sequence comprises a nucleotide sequence selected from the group consisting of the polynucleotide sequences as disclosed herein (e.g. SEQ ID Nos:1-3, SEQ ID Nos:19-47, SEQ ID NO:64, or as shown in FIG. 5, (e.g. SEQ ID NOs:65-85)).

In another aspect, the invention provides methods for expressing a gene of interest in a bacterial cell comprising: (a) obtaining a bacterial strain comprising a gene of interest operably linked to a heterologous promoter sequence for expression of the gene of interest in a bacterial cell, wherein the promoter sequence comprises a nucleotide sequence selected from the group consisting of the polynucleotide sequences as shown in FIG. 5, in SEQ ID NOs:64-85, in SEQ ID Nos:1-3, and SEQ ID Nos:19-47; (b) growing a culture of cells of the bacterial strain in the presence of an inducer of the heterologous promoter; and (c) assaying the culture, or a portion or an extract thereof, for expression of the gene of interest. The method may also comprise assaying a bacterial strain comprising such a construct by measuring the transformation frequency (“TF”) of a plant cell by the bacterial strain.

The mechanism of T-DNA transfer to plant cells by Agrobacterium has been well documented (e.g. Gelvin, Microbiology and Molecular Biology Reviews, 67:16-37, 2003). Briefly, the T-DNA is delimited by two border regions, referred to as right border (RB) and left border (LB). The borders are nicked by virulence protein VirD2 which produces single stranded transferred DNA (the “T-strand”) with covalent attachment of the VirD2 on its 5′ end. The protein-DNA complex, also including Agrobacterium VirE2 protein, exits Agrobacterium cells through the so-called Type 4 secretion system (T4SS, both virulence protein and ssDNA transporter), and is transferred into plant cells and integrated in the plant genome with the help of both Agrobacterium virulence proteins and plant factors.

The following descriptions and definitions are provided to better define the invention and to guide those of ordinary skill in the art in the practice of the invention. Unless otherwise noted, terms are to be understood according to conventional usage by those of ordinary skill in the relevant art.

“Amplifiers” are widely used devices to enhance signals in electrical and electronic systems. In synthetic biology, bio-amplifiers such as T7 RNAP and cooperative activator proteins (e.g., HrpR and HrpS) have also been used to increase the sensitivity and output dynamic range of transcription based biosensors (e.g. Tang et al., ACS Synthetic Biology 7:1753-1762, 2018 Wang et al., Nucleic Acids Research, 42:9484-9492, 2014). The transcriptional factor VirG of A. tumefaciens activates the expression of virulence genes by binding to “Vir boxes”, nucleotide sequences of the Vir operons in the presence of signals and the sensor protein VirA (Krishnamohan et al., J Bacteriol 183:4079-4089, 2001). In contrast, the VirG mutant VirG_{N54D} alone can activate the vir genes without signals and VirA (Jin et al., Molecular Microbiology, 7:555-562, 1993; Jung et al., Current Microbiology, 49:334-340, 2004). Thus an “amplifier” module may be constructed and utilized for enhanced or more precise control of gene expression. Such an amplifier module may comprise a promoter that functions in expression of a polynucleotide sequence of interest in a bacterial cell. Such a promoter may be a native (“wild-type”) promoter, or it may be modified or engineered to improve (increase or decrease) or otherwise control the resulting level of gene expression under certain growth conditions of interest. As used herein, “inducible promoter” refers to a promoter that exhibits an increased level of expression of an operably linked gene of interest, when cells comprising the promoter and the gene of interest are grown under inducing conditions, such as in the presence of a chemical or other inducer. Such “inducibility” may be due to direct or indirect effects as the inducer promotes gene expression.

As used herein, the term “recombinant” refers to a non-naturally occurring DNA, protein, cell, seed, or organism that is the result of genetic engineering and as such would not normally be found in nature. A “recombinant DNA molecule” is a DNA molecule comprising a DNA sequence that does not naturally occur in nature and as such is the result of human intervention, such as a DNA molecule comprised of at least two DNA molecules heterologous to each other. An example of a recombinant DNA molecule is a DNA molecule operably linked to a heterologous regulatory or other element, such as a heterologous promoter for expression in a plant cell, or other cell. A “recombinant protein” is a protein comprising an amino acid sequence that does not naturally occur and as such is the result of human intervention, such as an engineered protein or a chimeric protein. A recombinant cell, seed, or organism is a cell, seed, or organism comprising transgenic DNA, for example a transgenic cell, seed, plant, or plant part comprising a recombinant DNA molecule and therefore produced as a result of plant transformation.

As used herein, the term “genetic engineering” refers to the creation of a non-natural DNA, protein, or organism that would not normally be found in nature and therefore entails applying human intervention. Genetic engineering can be used to produce an engineered DNA, protein, or organism that was conceived of and created in the laboratory using one or more of the techniques of biotechnology such as molecular biology, protein biochemistry, bacterial transformation, and plant transformation. For example, genetic engineering can be used to express a gene of interest in a bacterial, fungal, plant, or animal cell.

The term “transformation frequency (“TF”) refers to the ability of a bacterial cell to transfer DNA via AMT, or other bacterial-mediated transformation. This may be measured, for instance, by the number of transformed cells or plants obtained from a given treated sample. Such transformation may be the result of transient or stable transformation.

The term “transgene” refers to a DNA molecule artificially incorporated into an organism's genome as a result of human intervention, such as a plant transformation method. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant or other organism is something distinctly different from the related wild-type plant or other organism and the transgenic trait is a trait not naturally found in the wild-type plant or other organism. Transgenic plants and organisms of the invention comprise the recombinant DNA molecules and engineered proteins provided by the invention.

As used herein, the term “heterologous” refers to the relationship between two or more things derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell, seed, or organism into which it is inserted when it would not naturally occur in that particular cell, seed, or organism.

As used herein, the term “protein-coding DNA molecule” refers to a DNA molecule comprising a nucleotide sequence that encodes a protein. A “protein-coding sequence” means a DNA sequence that encodes a protein. A “sequence” means a sequential arrangement of nucleotides or amino acids. The boundaries of a protein-coding sequence are usually determined by a translation start codon at the 5′-terminus and a translation stop codon at the 3′-terminus. A protein-coding molecule may comprise a DNA sequence encoding a protein sequence. As used herein, “transgene expression”, “expressing a transgene”, “protein expression”, and “expressing a protein” mean the production of a protein through the process of transcribing a DNA molecule into messenger RNA (mRNA) and translating the mRNA into polypeptide chains, which are ultimately folded into proteins. A protein-coding DNA molecule may be operably linked to a heterologous promoter in a DNA construct for use in expressing the protein in a cell transformed with the recombinant DNA molecule. As used herein, “operably linked” means two DNA molecules linked in manner so that one may affect the function of the other. Operably-linked DNA molecules may be part of a single contiguous molecule and may or may not be adjacent. For example, a promoter is operably linked with a protein-coding DNA molecule in a DNA construct where the two DNA molecules are so arranged that the promoter may affect the expression of the transgene.

As used herein, a “DNA construct” is a recombinant DNA molecule comprising two or more heterologous DNA sequences. DNA constructs are useful for transgene expression and may be comprised in vectors and plasmids. DNA constructs may be used in vectors for the purpose of transformation, that is the introduction of heterologous DNA into a host cell, in order to produce transgenic plants and cells, and as such may also be contained in the plastid DNA or genomic DNA of a transgenic plant, seed, cell, or plant part. As used herein, a “vector” means any recombinant DNA molecule that may be used for the purpose of bacterial or plant transformation. Recombinant DNA molecules as set forth in the sequence listing, can, for example, be inserted into a vector as part of a construct having the recombinant DNA molecule operably linked to a gene expression element that functions in a plant to affect expression of the engineered protein encoded by the recombinant DNA molecule. Methods for constructing DNA constructs and vectors are well known in the art.

The components for a DNA construct, or a vector comprising a DNA construct or expression cassette, generally include one or more gene expression elements operably linked to a transcribable DNA sequence, such as the following: a promoter for the expression of an operably linked DNA, an operably linked protein-coding DNA molecule, and a 3′ untranslated region (UTR). A promoter drives expression of the recombinant protein molecule. Gene expression elements useful in practicing the present invention also include, but are not limited to, one or more of the following type of elements: 5′ UTR, enhancer, leader, cis-acting element, intron, targeting sequence, 3′ UTR, and one or more selectable or screenable marker transgenes.

Promoters useful in practicing the present invention include those that function in a cell for expression of an operably linked polynucleotide, such as a bacterial promoter. The microbial genome is a useful source for identifying DNA segments such as promoters for synthetic biology applications (Jin et al., Applied Microbiology and Biotechnology, 103:8725-8736, 2019). Many endogenous promoters have been identified such as P_{sacB} promoter from B. subtilis THY-7 (Jin et al., 2019) and the P_{vgb} promoter from Vitreoscilla stercoraria (Lara et al., ACS Synthetic Biology, 6:344-356, 2017), in addition to, for instance, Pᵥᵢᵣ promoters found on the Agrobacterium Ti plasmid. Bacterial and plant promoters are varied and well known in the art and include those that are inducible, viral, synthetic, constitutive, temporally regulated, spatially regulated, and/or spatio-temporally regulated. The present invention further provides a panel of engineered (modified) bacterial promoter sequences for versatile application of an inducible bacterial gene expression system, such as the disclosed promoters that are controlled by VirG_{N54D}.

Recombinant DNA molecules of the present invention may be synthesized and modified by methods known in the art, either completely or in part, especially where it is desirable to provide sequences useful for DNA manipulation (such as restriction enzyme recognition sites or recombination-based cloning sites), or sequences useful for DNA construct design (such as spacer or linker sequences).

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar, Inc., 1228 S. Park St., Madison, Wis. 53715), and MUSCLE (version 3.6) (Edgar, Nucleic Acids Research 32(5):1792-7, 2004) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

The present invention includes recombinant DNA molecules and engineered proteins having at least 70% sequence identity, at least 80% sequence identity, at least 85% sequence identity, at least 90% sequence identity, at least 95% sequence identity, at least 96% sequence identity, at least 97% sequence identity, at least 98% sequence identity, and at least 99% sequence identity to any of the recombinant DNA molecule or polypeptide sequences provided herein. Such identity may be calculated over the full length of the protein or nucleotide sequence, or over a portion of the length of the sequences of interest, such as 1%, 5%, 10%, 25%, or 50% of the sequence length. Alternatively, identity may be calculated over a portion (“window”) of a sequence of interest based on nucleotide length such as 50 nucleotide base-pairs or amino acid residues, 100, 200, 500, 1000, 5000 etc., including intervening lengths. Variants having a percent identity to a sequence disclosed herein may have the same activity as the base sequence.

In one embodiment, fragments of a promoter sequence disclosed herein are provided. Promoter fragments may comprise promoter activity and may be useful alone or in combination with other promoters and promoter fragments, such as in constructing chimeric promoters, or in combination with other expression elements and expression element fragments. In specific embodiments, fragments of a promoter are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a recombinant DNA molecule disclosed herein. Fragments of a sequence disclosed herein may have the same activity as the base sequence. Methods for producing such fragments from a starting promoter molecule are well known in the art.

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including for instance angiosperms (monocotyledonous and dicotyledonous plants), gymnosperms, and ferns. Cells of other organisms may be of interest for instance to achieve improved transformation frequency (“TF”) by bacterial-mediated cell transformation, and may include for instance cells of fungi, algae, cyanobacteria, and animals such as nematodes, insects, fish, and mammals.

Exemplary plants contemplated herein may include monocotyledonous or dicotyledonous crop plants including, for instance, cassava, maize (corn; Zea mays), soybean (Glycine max), cotton (Gossypium hirsutum; Gossypium sp.), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp.); tall fescue (Festuca arundinacea); turfgrass species (e.g. species: Agrostis stolonifera, Poa pratensis, Stenotaphrum secundatum); wheat (Triticum aestivum); teff (Eragrostis); millet, alfalfa (Medicago sativa); members of the genus Brassica, including broccoli, cabbage, cauliflower, carrot, cucumber, dry bean and other leguminous plants, eggplant, tobacco (Nicotiana sp.), leek, lettuce, melon, okra, onion, pea, pepper, pumpkin, radish, spinach, squash, sweet corn, tomato, potato, watermelon, ornamental plants, and other fruit, vegetable, tuber, oilseed, and root crops, wherein oilseed crops may include soybean, canola, oil seed rape, oil palm, sunflower, olive, coffee, citrus, flaxseed, safflower, and coconut, among others. Host cells, such as Escherichia coli, and Agrobacterium sp. or other Rhizobia, comprising the disclosed constructs are also contemplated as part of the invention.

The resulting transgenic organisms such as plants, progeny, seeds, plant cells, plant parts, and/or cells of other contemplated organisms of the invention may contain or display one or more transgenic traits as a result of their genetic transformation. Other transgenic trait(s) may be introduced by co-transforming a DNA construct for that additional transgenic trait(s) with a DNA construct comprising the recombinant DNA molecules provided by the invention (for example, with all the DNA constructs present as part of the same vector used for plant transformation) or by inserting the additional trait(s) into a transgenic plant comprising a DNA construct provided by the invention or vice versa (for example, by using any of the methods of plant transformation on a transgenic plant or plant cell).

Transgenic traits include, but are not limited to, expression of a gene product of interest, increased insect resistance, increased water use efficiency, increased yield performance, increased drought resistance, increased seed quality, improved nutritional quality, hybrid or inbred seed production, male sterility, grain nutritional or energy value and herbicide tolerance, in which the trait is measured with respect to a wild-type plant. Such transgenic traits are well known to one of skill in the art.

Transgenic cells and progeny that contain a transgenic trait provided by the invention may be used with any breeding methods that are commonly known in the art. In plant lines comprising two or more transgenic traits, the transgenic traits may be independently segregating, linked, or a combination of both in plant lines comprising three or more transgenic traits. Back-crossing to a parental plant and out-crossing with a non-transgenic plant are also contemplated, as is vegetative propagation. Descriptions of breeding methods that are commonly used for different traits and crops are well known to those of skill in the art. To confirm the presence of the transgene(s) in cells of a particular organism such as a bacterial cell, plant cell, or seed, a variety of assays may be performed. Such assays include, for example, molecular biology assays, such as Southern and northern blotting, PCR, and DNA sequencing; biochemical assays, such as detecting the presence of a protein product, for example, by immunological means (ELISAs and western blots) or by enzymatic function; plant part assays, such as leaf or root assays; and also, by analyzing the phenotype of the whole plant, when identifying transformed cells.

Sequences for transgene expression may be codon optimized for expression in bacteria, algae, cyanobacteria, fungi, animals, or plants, including monocotyledonous and dicotyledonous plants. The genes of interest for expression may be located on the same construct, or on separate constructs, and may be co-transformed, transformed separately, or may be introduced together into a plant cell via a step of plant breeding. Marker-assisted selection may be utilized to confirm the presence of one or more gene(s) of interest via a plant breeding approach.

Stable or transient expression of constructs comprising a gene of interest is contemplated. The disclosure contemplates preparation of an expression vector that can be transported across a cell membrane, or a plant cell wall and membrane, resulting in transformation of a cell, for expression therein. Transformation of cells of organisms other than plants is also contemplated. In one embodiment, a vector may replicate in a bacterial host such that the vector can be produced and purified in sufficient quantities for transient expression or other use. In another embodiment, a vector can encode a marker gene to allow for selection or screening for the presence of the vector in a host cell such as a bacterial cell, an animal cell, an algal cell, a fungal cell, an insect cell, or a plant cell, or the vector can also comprise an expression cassette to provide for the expression of a gene of interest such as in a plant. The selection or marker gene may be expressed in a cell, or in a cell nucleus or in an organelle such as a chloroplast or a mitochondrion. In some embodiments an expression cassette contains a promoter region, a 5′ untranslated region, an optional intron to aid expression, and optionally a multiple cloning site to allow facile introduction of sequences of interest, and a 3′ UTR.

The method may further comprise assaying for the presence of an introduced gene in the genome of a cell, and/or the presence of a resulting protein product in the cell. Thus, well known methods such as Southern blotting and western blotting may be used. Methods may further comprise assaying for protein or enzyme activity. The presence of an introduced gene may be transient, or the gene may be stably integrated into a cell genome. Activity may thus be expressed in a transient or stable manner, and may occur in a cell, or in a cell nucleus, cytoplasm, mitochondria, or chloroplast.

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. Cited references are incorporated herein by reference. All such similar substitutes and modifications apparent to those skilled in the art are deemed to be within the spirit, scope and concept of the invention as defined by the appended claims.

### EXAMPLES

### Example 1: Strains and Cultivation Conditions

Strains and cultivation conditions. E. coli DH10B was the host for plasmid construction. The plasmid-free strain A. tumefaciens NTL4 (Luo, Z.-Q., et al.,. Mol. Plant-Microbe Interact. 14:98-103, 2001) was used as the host for promoter library construction and phenotypic validation. All strains were cultured in the Luria-Bertani (LB) with 200 rpm shaking at 37° C. (E. coli) or at 30° C. (A. tumefaciens). Appropriate antibiotics were added at the following concentrations (μg/mL): for A. tumefaciens, 100 of carbenicillin, 50 of kanamycin; for E. coli, 100 of ampicillin, 50 of kanamycin. Inducers of gene expression (cumic acid, L-arabinose, IPTG, vanillic acid, sodium salicylate, and naringenin) were added to the medium at the concentrations of 10-3-103 μmol/mL when necessary.

### Example 2: Plasmid Construction

Plasmids used in this study are listed in Table 1. Plasmids were constructed using the Gibson assembly method (Gibson et al., Nature Methods, 343-345. doi:10.1038/NMETH.13182009). All plasmids use pBBR1 (Szpirer et al., J Bacteriol. 183:2101-2110, 2001) or pVS1 (Vodala et al.,. Mol Cell. 31:104-13, 2008) as origins. To construct the inducible systems, the pBBR1 origin was cloned from the plasmid pBBR1-kan-hyg-ccdB (Wang et al., Nature Protocols 11:1175-1190, 2016). An ampicillin resistance cassette was cloned from the plasmid pAM PAT-ProCPCG. The sfgfp gene was cloned from the plasmid BP-Target-EndyD. The inducible systems were cloned from the plasmids pAJM.336 (Lad), pAJM.657 (cymR), 6 pAJM.661 (TtgR), pAJM.677 (AraE), pAJM.771 (NahR), and pAJM.773 (VanR) respectively (Meyer et al., 2019). The fragments were assembled to generate the plasmids pBBR1-B2, pBBR1-B4, pBBR1-B5, pBBR1-B7, pBBR1-B9, and pBBR1-B11. For the construction of the amplifier, VirG mutants virGNs4D were cloned from A. tumefaciens strains GV3101 and EHA105, respectively. Then virG_{N54D} mutants were introduced into the plasmid pBBR1-B5 to generate pBBR1-CN and pBBR1-BN accordingly. To construct the reporter, the sfgfp and mKate2 were cloned from the plasmid BP-Target-EndyD (Bonnet, et al, PNAS, 109:8884-8889, 2012). The virB and virE promoters were cloned from the strain EHA105. The pVS1 origin were cloned from the plasmid pCAMBIA5105 (Wendt, et al., Transgenic Research, 21:567-578, 2012). Then, these fragments were assembled to generate the plasmids pBVKGM. To screen promoters, fifteen promoters were cloned from the strain EHA105. The sfgfp gene was used as a reporter gene and the pVS1 was used as the origin. The constructed plasmids were named as pBVKP1-15. To construct pleD expressing plasmids, sfgfp from the plasmid pBVK-P3 was replaced by pleD to generate the plasmid pBVKpleD.

TABLE 1
Plasmids used in this study

| Plasmid | relevant properties | source or reference |
|---|---|---|
| pBBR1-kan-hyg-ccdB | Broad host range plasmid compatible with | Wang et al.,, Nature |
| | Inc. Q, Inc. P, Inc. W, and colE1; Kan^{R} | Protocols 11:1175- |
| | | 1190, 2016) |
| pBBR1-amp-B2 | CymR-Pcym-egfp, Amp^{R} | This study |
| pBBR1-amp-B4 | VanR-Pvan-egfp | This study |
| pBBR1-amp-B5 | LacI-Ptac-egfp, AmpR | This study |
| pBBR1-amp-B7 | araC-PBAD-egfp, AmpR | This study |
| pBBR1-amp-B9 | TtgR-Pttg-egfp, AmpR | This study |
| pBBR1-amp-B11 | NahR-Psal-egfp, AmpR | This study |
| PBBR1-Pcym-BN | CymR-Pcym-Bo542virGN54D, AmpR | This study |
| PBBR1-Pcym-CN | CymR-Pcym-C58virGN54D, AmpR | This study |
| PBBR1-Pcymcuo-CN | CymR-Pcymcuo-C58virGN54D, AmpR | This study |
| PBBR1-PBO-CN | CymR-PvirGBo542-C58virGN54D, AmpR | This study |
| PBBR1-Pc58-CN | CymR-PvirGC58-C58virGN54D, AmpR | This study |
| pCAMBIA5105 | pvs1 origin, KanR | Wendt et al., 2012 |
| BP-Target-EndyD | egfp and mkate2 | Lab stock |
| pBVK-GFP-mKate | PvirB-egfp; PvirE-mkate2, | This study |
| pBVK-P1 | PvirA-egfp, KanR | This study |
| pBVK-P2 | PvirG-egfp; KanR | This study |
| pBVK-P3 | PvirB-egfp, KanR | This study |
| pBVK-P4 | PvirC-egfp; KanR | This study |
| pBVK-P5 | PvirD-egfp, KanR | This study |
| pBVK-P6 | PvirE-egfp, KanR | This study |
| pBVK-P7 | PvirH1-egfp, KanR | This study |
| pBVK-P8 | PvirH2-egfp, KanR | This study |
| pBVK-P9 | PvirM-egfp, KanR | This study |
| pBVK-P10 | PvirP1-egfp, KanR | This study |
| pBVK-P11 | PvirP2-egfp, KanR | This study |
| pBVK-P12 | Prep-egfp, KanR | This study |
| pBVK-P13 | PBO100-egfp, KanR | This study |
| pBVK-P14 | PBO114-egfp, KanR | This study |
| pBVK-P15 | PBO210-egfp, KanR | This study |
| pBVK-pled | PvirB-pled, KanR | This study |
| pBVK-HH | PvirB-egfp, PvirB75-mkate2, KanR | This study |
| pBVK-HL | PvirB-egfp, PvirB66-mkate2, KanR | This study |
| pBVK-LL | PvirB21-egfp, PvirB66-mkate2, KanR | This study |
| pBVK-pled-Hgfp | PvirB-pled, PvirB75-egfp | This study |
| pBVK- pled-Lgfp | PvirB-pled, PvirB21-egfp | This study |

### Example 3: GFP and Congo Red Assays

GFP measurement. An inoculum of A. tumefaciens was grown overnight to stationary phase and then transferred to fresh media at a 1:100 dilution. The new inocula were added with inducers to a final concentration of 1×10⁻³ to 1×10³ μmol/ml when their optical density at 600 nm (OD₆₀₀) reached 0.5. After 6 h of incubation, cells were collected and resuspended in 1×PBS buffer. Then 100 μL of suitably diluted cultures were added into 96-well microtiter plates. OD600 and relative green fluorescence were measured by a BioTek® microplate reader H1. Congo red plate assay. The dye was dissolved in ddH2O at 20 mg ml⁻¹ and passed through 0.2 μm filters immediately. Four ml of filtered Congo Red was added per L (final concentration: 80 μg/ml) to generate LB-CR agar medium. The overnight cultured cultures were diluted to an OD₆₀₀ of 1.0. 10 μl cultures were plated on LB-CR agar with appropriate antibiotics, followed by incubation at 30° C. for 24 hours.

### Example 4: Cultivation and Analysis of Static Biofilms

Biofilms were determined using the sterile coverslip cultured method with minor modifications (Xu et al., Molec. Microbiol. 89:929-948, 2013). Briefly, for biofilm formation, 18 mm glass coverslips were added into the 12-well polystyrene cell culture plates. One ml of pre-cultured cells were inoculated into the plates at an OD₆₀₀ of 0.05, and incubated without shaking for 24 h at 30° C. To quantify biofilm formation the culture supernatants were removed and the coverslips were washed twice in ddH₂O. The remaining attached bacteria were stained by 0.1% (w/v) crystal violet for 10 min and washed twice in ddH₂O. Biomass adhering to the coverslip was extracted with 1 ml of 33% acetic acid to solubilize the biofilm. The optical density (OD) of 150 μl of dilution cultures was measured at OD₅₉₅ nm (A595) in a microplate reader.

### Example 5: Construction and Characterization of Inducible Expression Systems in A. Tumefaciens

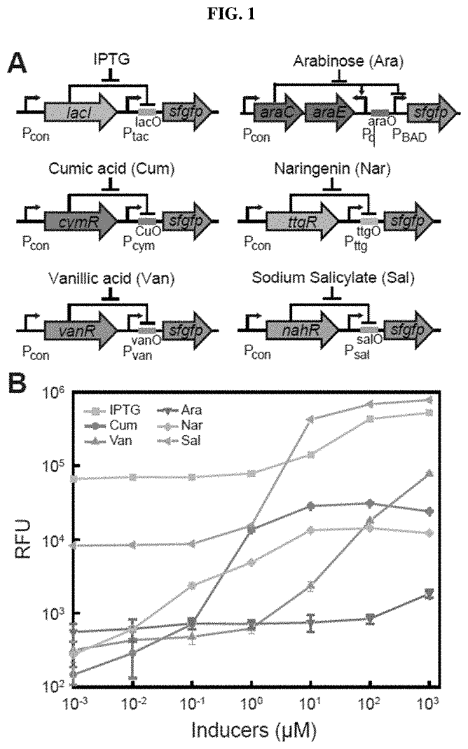

A reliable induction system is required to achieve precise control of gene expression. To develop effective tools for A. tumefaciens, six candidate inducible expression systems (the β-d-1-thiogalactopyranoside (IPTG-), cumic acid (Cum-), vanillic acid (Van-), arabinose (Ara-), naringenin (Nar-), and sodium salicylate (Sal-) inducible systems) were chosen for study. These systems exhibit low background expression and a large dynamic range in E. coli (Meyer et al., Nature Chemical Biology, 15:196-204, 2019). An ori from the broad host-range plasmid pBBR1, which propagates in both E. coli and A. tumefaciens, was utilized for the origin of replication (Szpirer et al., J Bacteriol. 183:2101-2110, 2001). To evaluate the performance of these inducible systems, a green fluorescence protein gene (sfgfp) was utilized as a reporter gene, and placed under the control of the corresponding inducible promoters, P_{tac}, P_{cym}, Pᵥₐₙ, P_{BAD}, P_{ug} and Pₛₐₗ, respectively. The repressor genes of the inducible systems (lacI, cymR, vanR, araE, ttgR and nahR) were driven by a common constitutive promoter (P_{con}). Schematic diagrams of these inducible systems are shown in FIG. 1A. The resulting inducible “expression circuits” were transformed into A. tumefaciens NTL4.

To evaluate these induction systems, green fluorescence intensities of cells transformed with the plasmids were measured at various concentrations of inducers, ranging from 10⁻³ to 10³ μM. As shown in FIG. 1B, all six systems showed sigmoidal response in A. tumefaciens. At the highest induction concentration (i.e., 10³ μM), the expression levels of the six systems followed the following high-to-low order: Sal, IPTG, Van, Cum, Nar and Ara. At the lowest induction concentration (10⁻³ μM), the observed expression levels from low to high were: Cum, Van, Ara, Nar, Sal, and IPTG. Notably, strains with Sal- and IPTG-inducible systems had the highest expression levels of 7.8×10⁵ and 5.2×10⁵ respectively; however, their basal expression was also high (8.3×10³ and 6.6×10⁴). The Nar- and Ara-inducible systems were low in their highest expression levels (1.2×10⁴ and 1.8×10³) while their basal expression levels remained relatively high (1.8×10² and 5.5×10²). By contrast, the Van- and Cum-inducible systems showed both high expression levels (7.7×10⁴ and 3×10⁴) and low expression levels (3.1×10² and 1.4×10²) at highest and lowest inducer concentrations respectively, offering both a large dynamic range and lower leakiness. The Cum-inducible system was selected as an induction module to advance development of synthetic biology tools for A. tumefaciens.

### Example 6: Expression Amplification by Introducing VirG_{N54D} into the Cumic Acid-Inducible System

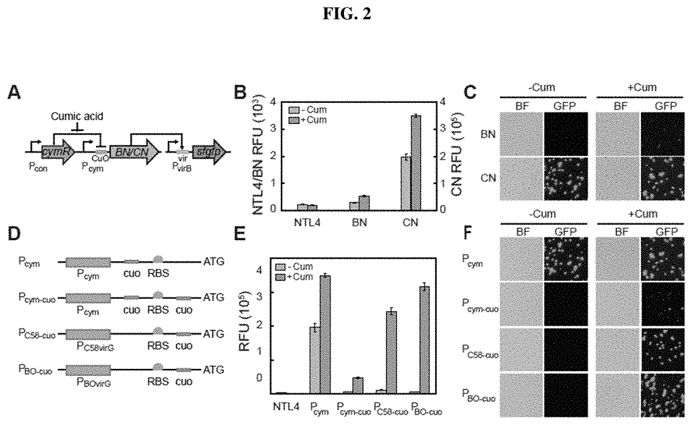

An “amplifier module” was constructed to increase controllable expression in A. tumefaciens. To enable expression amplification, two VirG variants from the plasmids pTi_{C58} and pTi_{BO542} were utilized. These were designated CN and BN respectively, and inserted under the control of the cumic acid-inducible promoter P_{cym} (FIG. 2A). To verify their functionality VirG variants were used to drive the reporter gene sfgfp via the VirG-controlled promoter P_{virB} (FIG. 2A). FIG. 2B-2C show that both CN and BN successfully activate sfgfp expression in the presence of cumic acid. CN exhibited a much stronger activation of sfgfp expression than BN (3.5×10⁵ vs 5.4×10³), and was thus selected for subsequent use in constructing the amplifier. However, induction of CN resulted in an increased level of leakiness of expression in the absence of inducer, due to strong amplification. To address this issue, three new cumic acid-inducible promoters were constructed with reduced leakiness: P_{cym-cuo}, P_{C58-cuo} and P_{BO-cuo}, as shown in FIG. 2D. The P_{cym-cuo} promoter was obtained by adding one additional cymR-binding site (cuo) between the ribosome binding site and the start codon ATG. The P_{C58-cuo} and P_{BO-cuo} promoters were constructed by introducing the cymR binding site into the weak virG promoters from the plasmids pTi_{C58} and pTi_{BO542} respectively. As depicted in FIG. 2E and FIG. 2F, all three engineered promoters have significantly decreased basal sfgfp expression when compared with the promoter P_{cym}. Among the three, the promoter P_{BO} showed a minimal leakiness while maintaining a high level of induced expression level, and was integrated with CN to form a further optimized amplification module.

### Example 7: Identification and Evaluation of VirGN54D-Controlled Promoters

To enable versatile applications of inducible gene expression system, promoters that are controlled by VirG_{N54D} in the amplification module were identified. MEME, a program for ab initio identification of novel motifs (Bailey et al., Nucleic Acids Research, 37(suppl_2), W202-W208, 2009), was utilized to identify VirG-controlled promoters in the upstream regions of all upregulated genes in the plasmid pTiBo542. The consensus VirG-binding motif, RTTDCAWWTGHAAY (SEQ ID NO:47), with up to three mismatches allowed (Haryono et al., Frontiers In Microbiology, 10:1554, 2019) was used in the search, which resulted in 15 putative promoters as shown in FIG. 3A (SEQ ID NOs:48-62).

Notably, the promoters of virA, virB, virC, virD, virE, virG, and repABC operons have the consensus VirG-binding motif, consistent with the previous reports that these genes are activated by VirG (Cho & Winans, PNAS 102:14843-14848, 2005). Other putative promoters PBO100, PBO114, and PBO210 also contain the VirG-binding motif, indicating that these promoters may also be controlled by VirG.

To validate these promoters they were cloned from the plasmid pTi_{BO542} and placed upstream of sfgfp for fluorescence-based quantification. The results (FIG. 3B) show that these promoters have distinct expression characteristics and can be divided into three groups, namely strong, medium, and weak promoters. P_{virB}, P_{VirPI}, P_{virP2}, P_{virE}, and P_{BO210} are strong promoters; P_{BO114}, P_{virH2}, P_{virH1} and P_{BO100} are medium promoters; P_{virD}, P_{virA}, P_{virC}, Pᵣₑₚ, P_{virG} and P_{virM} are weak promoters. Among these promoters, P_{virB} exhibited the highest induced level and a minimal basal expression, and was selected for further optimization in view of its wide dynamic range of expression.

### Example 8: Gene Expression Fine-Tuning with Altered Spacer Sequences

Complex biosynthetic pathways may require a coordinated, fine balance of expression of individual genes in order to achieve optimal performance. Thus strategies were developed for gene expression fine-tuning in A. tumefaciens. Using simple sequence repeats in the spacer region between the ribosome-binding site and the start codon (ATG) is a simple and effective approach to tune gene expression in E. coli (Egbert & Klavins, PNAS 109:16817-16822, 2012).

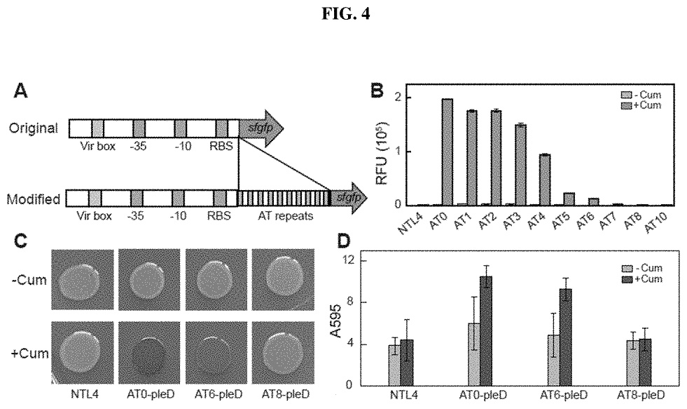

To test the feasibility of this approach for modulating translation in A. tumefaciens, various lengths of AT sequence repeats ((AT)₀-(AT)₁₀) were inserted in the spacer region between the promoter P_{virB} and the fluorescence reporter gene sfgfp (FIG. 4A, and SEQ ID Nos:19-26). Fluorescence output of strains carrying the plasmids P_{virB-(AT)n}-sfgfp P with different repeats was then measured. As shown in FIG. 4B, the fluorescence intensity decreased monotonically as the number of AT repeats increased.

The results showed that altering AT repeats can robustly and predictably tune gene expression levels over a 100-fold range. To demonstrate the application of this fine-tuning strategy, three constructs, P_{virB-(AT)0}, P_{virB-(AT)6} and P_{virB-(AT)8} were to drive the expression of pleD which encodes the protein that positively regulates UPP polysaccharide synthesis and biofilm formation in A. tumefaciens (Hengge, Nature Rev. Microbiol. 7:263-273, 2009; Xu et al., Molec. Immunol. 89:929-948, 2013). Strains carrying with the plasmids P_{virB-(AT)0}-PleD, P_{virB-(AT)6}-pleD, and P_{virB-(AT)8}-pleD and tested their polysaccharide production. Visible Congo Red dye staining was used to evaluate the production of polysaccharides, because the intensity of red staining is proportional to the amount of Congo Red-reactive polysaccharide produced (Xu et al., 2013). As shown in FIG. 4C, the colony of the A. tumefaciens cells of the strain harboring the P_{virB-(AT)0}-pleD plasmid resulted in a stronger red staining in the presence of cumic acid than in the absence of the inducer, demonstrating that the production of PleD elevates polysaccharide production in A. tumefaciens. In addition, by comparing the colony staining of the strains carrying P_{virB-(AT)0}-pleD, P_{virB-(AT)6}-pleD, and P_{virB-(AT)8}-pleD, the intensity of red staining decreased monotonically with the increase of the AT repeat number. This finding was further supported through a colorimetric, quantitative comparison as shown in FIG. 4D. These results demonstrated that using AT repeats is a feasible method to tune the translational rate and hence to precisely control gene expression.

### Example 9: Construction of a Promoter Library for Gene Expression Fine-Tuning

Site-specific mutagenesis of promoters is another powerful strategy to fine-tune gene expression (Qin et al., Appl Environ Microbiol. 77:3600-3608, 2011). As shown in FIG. 3, the fifteen identified native VirG-controlled promoters have distinct activities and are also significantly different in terms of the sequence of their VirG binding sites. Therefore, the VirG binding site sequence may play a key role in controlling promoter activity.

To test this possibility, the promoter Persi was utilized as a template and mutations were introduced into its binding site while conserving the core region (CAATTG; SEQ ID NO:63) and randomizing other sites, yielding RYTNCAATTGNAAY (SEQ ID NO:64; R=A or G; Y=C or T; N=A, T, G or C) (FIG. 5A). To screen the randomized promoters efficiently, sfgfp was used as a reporter gene and placed under the control of the mutated promoters. 300 colonies were selected and grown in LB medium with a supplement of 100 μM of cumic acid. The resulting mutant strains were measured with a microplate reader to determine their promoter strength. Finally, 21 mutants with different strengths that span across the expression spectrum were selected (FIG. 5C; SEQ ID Nos:65-85). The corresponding sequences of the mutated binding sites are shown in FIG. 5C, with mutated base pairs highlighted. These results demonstrated that random mutation of the VirG binding site is an efficient method to alter gene expression by tuning transcription.

### Example 10: Demonstration of the Gene Expression Toolkit

Gene clusters such as those produce complex metabolic pathways often involve multiple genes that are expressed at different levels for an optimal realization of function. To demonstrate the utilization of the described controlled gene expression toolkit, the feasibility of using a single inducer to simultaneously regulate multiple genes with different expression was studied. The virB promoter library, the cumic acid-based induction system, and the CN-based amplification module was utilized to control three pairs of PvirB promoter variants with differential activities for driving the expression of the two fluorescence reporter genes, sfgfp and mkate2.

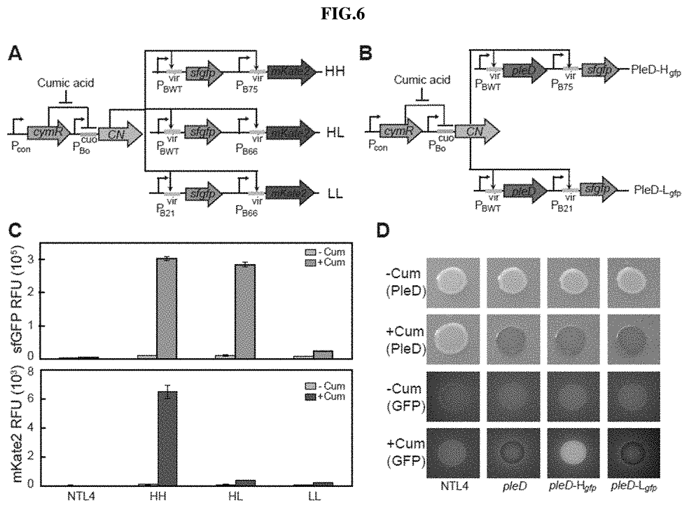

As shown in FIG. 6A, in the first pair (HH), both sfgfp and mkate2 were driven by strong promoters (P_{BWT} and P_{B75} respectively). In the second pair (HL), sfgfp was controlled by the strong promoter P_{BWT} and mkate2 was controlled by the weak promoter P_{B66}. In the third pair LL, both sfgfp and mkate2 were regulated by weak promoters P_{B21} and P_{B66}, respectively. As shown in FIG. 6B, the strain harboring the plasmid with two strong promoters (i.e., pHH) exhibited a high expression level of sfgfp and mkate2 expression upon induction of the single signal (cumic acid). The strain harboring the plasmid pHL exhibited a high expression level of sfgfp but a low level of mKate2. The strain harboring pLL exhibited lower levels of both sfgfp and mkate2.

To further illustrate application of the toolkit two sets of controllable expression systems to simultaneously drive pleD and sfgfp (FIG. 6C) were utilized. In the former expression is controlled by the promoter PBwt while in the latter expression is regulated by a strong promoter (P_{B75}) or a weak promoter (P_{B21}). The strain containing the construct with P_{BWT} and P_{B75} showed both strong Congo red staining and strong green fluorescence in the presence of cumic acid. In contrast, the strain with P_{BWT} and P_{B21} showed only strong red colony staining without green fluorescence. For comparison, the control strain (NTL4) did not show either red staining or green fluorescence and the strain driving pleD alone (i.e., the strain carrying the plasmid P_{BWT}-pleD) was able to produce Congo red. These results demonstrate that using the toolkit, such as with P_{virB} promoter variants can confer simultaneous differential control of multiple genes in a predictable desired manner.

1. A recombinant polynucleotide construct comprising a DNA molecule encoding:
a. at least one gene of interest operably linked to a heterologous inducible promoter for expression of the gene of interest in a bacterial cell, wherein the ratio of expression of the gene of interest in the presence of an added inducer relative to expression in the absence of the added inducer is at least 100; and
b. a broad host range origin of replication functional in Enterobacteriaceae and Rhizobiaceae.

2. The construct of claim 1, wherein the inducer is cumic acid or vanillic acid.

3. The construct of claim 1, wherein the origin of replication comprises an oriT functional with IncQ, IncP, IncW, or colE1.

4. The construct of claim 1 wherein the ratio of expression of the gene of interest in the presence of an added inducer relative to expression in the absence of the added inducer is at least 200, 300, 400 or 500.

5. A transgenic bacterium comprising the recombinant polynucleotide construct of claim 1.

6. The bacterium of claim 5 wherein the bacterium is from a species within a genus selected from the group consisting of: Escherichia, Agrobacterium, and Rhizobium.

7. The bacterium of claim 6, wherein the bacterium is an Agrobacterium tumefaciens bacterium or an Agrobacterium rhizogenes bacterium.

8. An in vitro culture of the bacterium of claim 5 growing in the presence of an inducer.

9. A culture of the bacterium of claim 5 growing in the presence both of a plant cell and of the inducer.

10. The bacterium of claim 5, further comprising a VirG_{N54D} protein.

11. The bacterium of claim 5, wherein the heterologous inducible promoter comprises a nucleotide sequence selected from the group consisting of: SEQ ID Nos:1-3, SEQ ID Nos:19-26, and SEQ ID Nos:27-47.

12. The bacterium of claim 5, wherein the heterologous inducible promoter comprises a nucleotide sequence selected from the group consisting of: SEQ ID Nos:64, and 65-85.

13. A method for expressing a gene of interest comprising:
a. obtaining the bacterium of claim 12;
b. growing a culture of cells of the bacterium in the presence of an inducer of the heterologous promoter; and
c. assaying the culture, or a portion or an extract thereof, for expression of the gene of interest.

14. The method of claim 13, wherein the culture of the bacterium further comprises plant cells.

15. The method of claim 13, wherein the assaying comprises measuring the transformation frequency (“TF”) of a plant cell by the bacterium.

16. A polynucleotide construct comprising a gene of interest operably linked to a heterologous inducible promoter sequence for expression of the gene of interest in a bacterial cell, wherein the promoter sequence comprises a nucleotide sequence selected from the group consisting of: SEQ ID Nos:64-85.

17. A kit comprising the bacterium of claim 12 and an inducer of the heterologous promoter.
